(19) United States
(12) Patent Application Publication
(10) Pub. No.: US 2005/0228312 A1
(43) Pub. Date: Oct. 13, 2005

(54) Biopsy needle system

(76) Inventor: Vihar Surti, Winston-Salem, NC (US)

Correspondence Address: BRINKS HOFER GILSON & LIONE/CHICAGO/COOK, PO BOX 10395, CHICAGO, IL, 60610, US

(21) Appl. No.: 11/095,156

(22) Filed: Mar. 30, 2005

Related U.S. Application Data
(60) Provisional application No. 60/557,970, filed on Mar. 31, 2004.

(51) Int. Cl. A61B 10/00; A61B 18/04

(52) U.S. Cl. 600/567; 600/564; 606/27

(57) ABSTRACT

A needle system is disclosed and a method for performing fine needle aspiration of a target tissue is disclosed. The disclosed needle system may be used during fine needle aspiration of a target tissue that is obstructed or partially obstructed by a tumor or other lesion by heating the needle system prior to advancing the needle system through the tumor or other lesion and subsequently advancing the heated needle system through the tumor or other lesion. This method may substantially prevent the seeding of cancerous cells during fine needle aspiration.

This application claims priority to U.S. Provisional Application Ser. No. 60/557,970, filed Mar. 31, 2004.

### BACKGROUND

This invention relates generally to medical devices, and more particularly to medical devices and methods for sampling targeted lymph nodes or other masses obstructed by a tumor.

Fine needle aspiration, or FNA, is a procedure that generally uses a fine gauge needle and an actuating device to sample tissue from, for example, a lymph node. A lymph node is a rounded mass of lymphatic tissue that is surrounded by a capsule of connective tissue. Lymph nodes filter lymph (lymphatic fluid), and they store lymphocytes (white blood cells).

The needles used during FNA typically range from 22 to 27 gauge. Generally, during FNA, the needle is inserted through the tissue layers of a patient and into the lymph node or other mass such as, for example, a lump in the breast or other area of a patient. FNA has broad uses and may be used in conjunction with an endoscope for performing FNA in the gastrointestinal or other internal areas of a patient, or may be used percutaneously by inserting the needle into, for example, a breast to sample a lesion in an area of a patient's body. Generally, negative pressure is created in a syringe or another sample gathering device and, as a result of the pressure difference between the syringe and the mass, cellular material can be drawn into the syringe through the needle or the needle system. Alternatively, the sample may be housed within a lumen of a needle during retraction of the needle system from the patient and subsequently gathered for storage or analysis by other means. FNA is generally accurate and frequently prevents the patient from having an open, surgical biopsy, which is more painful and costly.

However, this procedure becomes complicated if the target lymph node or target tissue is obstructed by tumor growth. In such circumstances, the needle must sometimes be passed through the tumor growth to reach the target lymph node or target tissue. This could result in the spreading of cancer cells within a patient. For example, if the needle is inserted through the tumor growth en route to the target lymph node or target tissue, cancer cells within the tumor may be pushed into other areas of the patient as a result of the needle intrusion. This is sometimes referred to as seeding of cancer cells.

Therefore, it would be advantageous if a device could, during FNA, go directly through tumor growth that obstructs a target lymph node in a way that substantially prevents the seeding of cancerous cells to other areas of the patient.

### SUMMARY

Disclosed herein is a needle system that substantially prevents the seeding of tumor cells during fine needle aspiration. By applying heat to the needle system prior to passing through a tumor cell that, for example, is obstructing a lymph node, the needle system cauterizes a path through the tumor cell thereby preventing the seeding of tumor cells to other areas of the patient.

The needle system may include an inner handle member and an outer handle member. The needle system may also include an elongate sheath attached to the inner handle member and extending beyond the distal end of the inner handle member. The needle system may also include an outer needle attached to the outer handle member and extending through the sheath, as well as an inner needle attached to the inner handle member. A stylet may also be included as well has a means for heating the needle system.

The needle system may be used in conjunction with an endoscope by connecting the needle system to the endoscope channel or, for example, used during endoscopic ultrasonography. For example, the needle system disclosed herein may include a plurality of indentations that enable the operator to take advantage of ultrasound principles while advancing the needle system through the tissues of a patient during the biopsy procedure.

Also disclosed herein is a method for performing fine needle aspiration on a target tissue, including providing a needle system and applying heat to the needle system prior to passing through a tumor or other lesion. The method also may include advancing the heated needle system through the tissue layers of the patient and through the tumor or other lesion, cooling the needle system, and then collecting a sample from a target tissue.

The present invention, together with further objects and advantages, will be best understood by reference to the following detailed description taken in conjunction with the accompanying drawings.

### BRIEF DESCRIPTION OF SEVERAL VIEWS OF THE DRAWINGS

FIG. 1 is a perspective view of a biopsy needle system according to one embodiment of the invention;

FIG. 2 is a perspective view of the biopsy needle system illustrated in FIG. 1 shown in an open configuration;

FIG. 2A is a perspective view of the biopsy needle system illustrating another embodiment of the needle system;

FIG. 2B is a perspective view of the biopsy needle system illustrating the embodiment illustrated in FIG. 2A;

FIG. 3 is a longitudinal sectional view of the handle portion of the biopsy needle system illustrated in FIG. 1;

FIG. 4 is a longitudinal sectional view of the distal end of the biopsy needle system illustrated in FIG. 1;

FIG. 5 is a cross-sectional view of the biopsy needle system taken along line 5-5 of FIG. 1;

FIG. 6 illustrates an alternative embodiment of a biopsy needle system with a plurality of indentations; and

FIG. 7 illustrates the biopsy needle system of FIG. 1 being used to perform fine needle aspiration.

### DETAILED DESCRIPTION OF THE PREFERRED EMBODIMENTS

The following provides a detailed description of several embodiments of the invention. The embodiments described and illustrated herein are exemplary in nature, and are not intended to limit the scope of the invention in any matter. Rather, the description of these embodiments serves simply to aid in enabling one of ordinary skill in the art to make and use the invention.

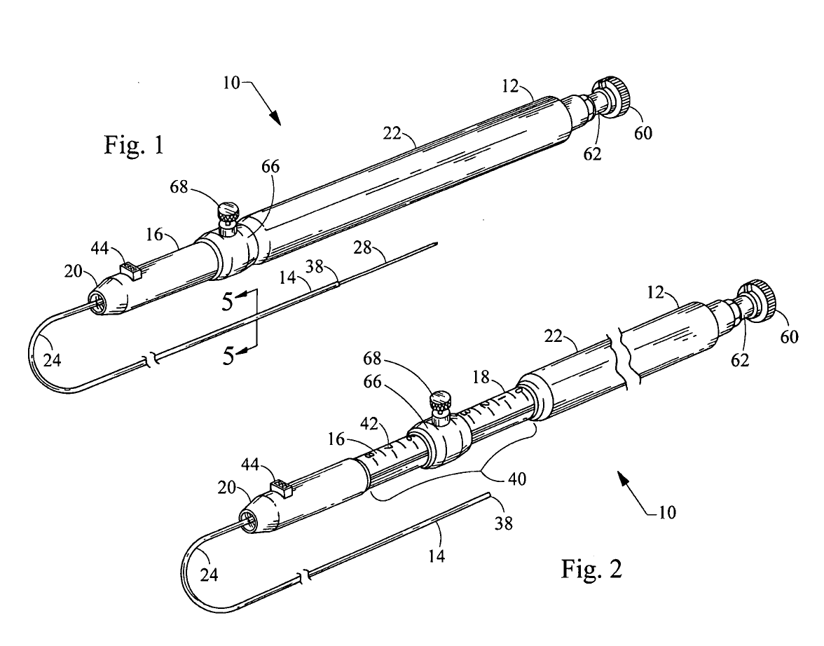

FIGS. 1-5 illustrate an exemplary biopsy needle system 10 having a proximal end 12 and a distal end 14. In this embodiment, the needle system 10 includes an inner handle member 16 having proximal 18 and distal 20 ends. An outer handle member 22 is movably disposed on the proximal 18 end of the inner handle member 16. An elongate sheath 24 is attached to the inner handle member 16 and extends axially beyond the distal end 20 of the inner handle member 16. As used herein, the term “axially” refers to one member situated around, in the direction of, on, or along an axis of another member, and is not limited to one member situated around, in the direction of, on, or along the central axis of another member.

As best seen in FIG. 4, the sheath 24 defines a sheath lumen 26 disposed therein. An outer needle 28, inner needle 30, and stylet 32 all may be attached to the outer handle member 22 and at least partially disposed in the sheath lumen 26. The outer needle 28 defines an outer needle lumen 34 and the inner needle 30 defines an inner needle lumen 36.

FIG. 1 illustrates the needle system 10 in a closed configuration. That is, the outer handle member 22 is advanced fully onto the respective portion of the inner handle member 16. In this configuration, the outer handle member 22 envelopes the inner handle member 16.

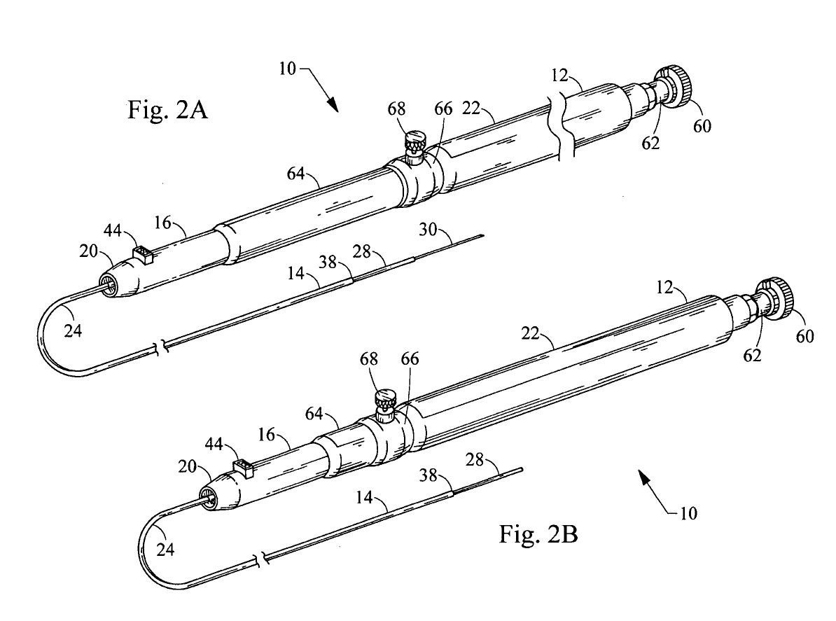

FIG. 2 illustrates the needle system 10 in an open configuration. In this configuration, the outer handle member 22 is retracted away from the distal end 20 of the inner handle member 16. This configuration more fully reveals the inner handle member 16. Actuating the needle system 10 from the closed configuration illustrated in FIG. 1 to the open configuration illustrated in FIG. 2 results in different relative positions of various components. For example, in the closed position, the outer needle 28 is shown axially extended from a distal end 38 of the sheath 24 (see FIG. 1). However, when the needle system 10 is in the open configuration, the outer needle 28 will be retracted axially within the sheath 24 (see FIG. 2). Also, because the inner needle 30 and the stylet 32 may be attached to the outer handle member 22, when the outer needle 28 is in the open configuration, the inner needle 30 and the stylet 32 will also be retracted axially within the sheath 24. In other words, a comparison of FIGS. 1 and 2 illustrates that the outer needle 28, with the inner needle 30 and the stylet 32 disposed therein, may extend axially beyond the distal end 38 of the sheath 24 when in a closed configuration (FIG. 1), but may be completely within the sheath 24 when the needle system 10 is in the open configuration (FIG. 2).

The needle system 10 can further include various adaptations to facilitate operation of the needle system 10. For example, as best illustrated in FIG. 2, a series 40 of indicia or gradations 42 can be disposed on the inner handle member 16. In particular, the series 40 can be disposed on a portion of the inner handle member 16 along which the outer handle member 22 is moved. In this configuration, each gradation 42 of the series 40 can correspond to a predetermined position of the outer needle 28 with the inner needle 30 and the stylet 32 retracted therein. The outer needle 28 is attached to the outer handle member 22. Furthermore, each gradation 42 of this series 40 can correspond to a predetermined length by which the outer needle 28, inner needle 30 and the stylet 32 extend axially beyond the distal end 38 of the sheath 24.

A further comparison of FIGS. 1 and 2 illustrates an example of the operation of this series 40 of gradations 42. In the open configuration illustrated in FIG. 2, the proximal most gradation viewable in the series 42 is “0”. Also in this configuration, the outer needle 28, inner needle 30 and the stylet 32 do not extend beyond a distal end 38 of the sheath 24. Thus, in this example, the gradation “0” can correspond to a zero length of the outer needle 28, inner needle 30 and the stylet 32 that extends axially beyond a distal end 38 of the sheath 24. In FIG. 1, the needle system 10 is in a completely closed configuration. To achieve this configuration from the open configuration illustrated in FIG. 2, a user would advance the outer handle member 22 over the entire series 40 of gradations 42. As the user moves the outer handle member 22 along the inner handle member 16, the outer handle member 22 successively passes gradations 42 of the series 40. Each gradation 42 can correspond to a length by which the outer needle 28, inner needle 30 and the stylet 32 extends beyond the distal end 38 of the sheath 24. Once the outer handle member 22 is fully advanced over the inner handle member 16, the entire series 40 of gradations 42 is covered. As illustrated in FIG. 1, this can correspond to a maximum length by which the outer needle 28 extends beyond the distal end 38 of the sheath 24. Thus, by moving the distal end of the outer handle member 22 to a specific gradation 42, a user of the needle system 10 can advance the outer needle 28, inner needle 30 and the stylet 32 to a desired position relative to the sheath 24. To aid in the accuracy of the needle system 10 articulations, a position ring 66 may be included. The position ring 66 may be moved slidably along the series 40 and positioned at a desired gradation 42. Then, the position ring fastener 68 may be tightened to restrict movement of the position ring 66. This allows a user to slide the outer handle member 22 until it is stopped by the by the position ring 66, thereby providing more accuracy in the articulation of the various components of the needle system 10.

The inner needle 30 and the stylet 32 can further move relative to the outer needle by extending axially beyond the outer needle 28. This movement of the inner needle 30 relative to the outer needle 28 can occur if the inner needle 30 is attached to the inner handle member 16. A user can extend the inner needle 30 axially from the outer needle 30 by, for example, twisting the distal end 20 of the inner handle member. Alternatively, for example, and as shown in FIGS. 2A and 2B, the needle system 10 may include an intermediate handle member 64 that is slidably disposed on the inner handle member 16. In this embodiment, the outer handle member 22 is slidably disposed on the intermediate handle member 64. The inner needle 30 may be attached to the intermediate handle member 64. The inclusion of an intermediate handle member 64 allows for the outer needle 28, inner needle 30 and the stylet 32 to extend axially beyond the distal end 38 of the sheath 24 when the outer handle member 22 is moved relative to the inner handle member 16 (FIG. 2B). However, when the outer needle 28 is axially extended, the intermediate handle member 64 may then be moved relative to the inner handle member 16 and distally from the outer handle member 22 such that the inner needle 30 and the stylet 32 axially extend further beyond a distal end of the outer needle, as shown in FIG. 2A.

Other embodiments, configurations and components of the needle system 10 that facilitate the relative movement of the interior components of the device are disclosed in co-pending application Ser. No. 10/699,487, which is hereby incorporated by reference.

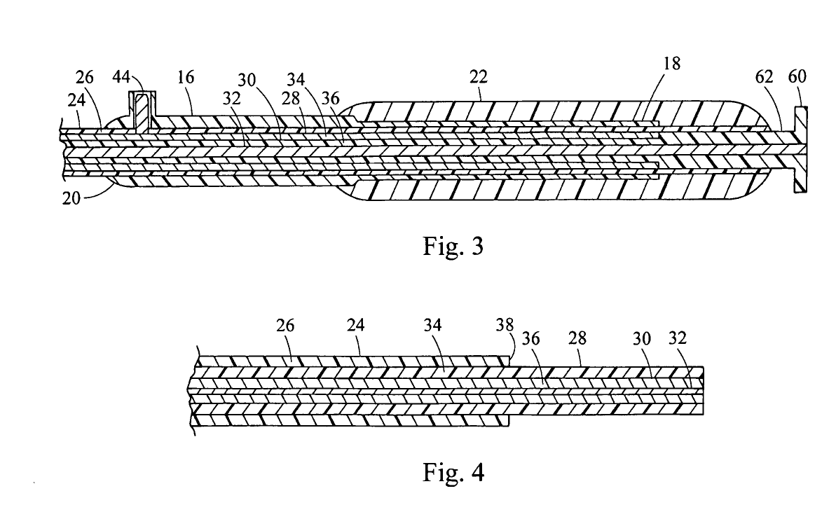

In FIGS. 3 and 4, the stylet 32 is shown slidably extended through the inner needle lumen 36. The stylet 32 acts to prohibit undesired material from gathering in the inner needle lumen 36 while the needle system 10 is advanced through the tissues of a patient. When a sample is to be taken, the stylet 32 may be withdrawn towards the proximal end 12 of the needle system 10 such that the inner needle lumen 36 may collect material upon further advancement of the inner needle 30 through the tissues of a patient. The withdrawing of the sylet 32 is accomplished by the stylet being attached to a stylet cap 60, which is disposed on a connector 62 located near the distal end 12 of the needle system 10. To withdraw the stylet 32, the operator simply pulls the stylet cap 60 away from the distal end 12 of the needle system 10. Upon removal of the stylet cap, the connector 62 will be exposed. The connector 62 is configured to accept various instruments that may aid in the gathering of a sample, such as a syringe.

Moreover, the distal end 20 of the inner handle member 16 has the ability to be in contact with an endoscope or other medical device. In such a configuration, the sheath 24 may be inserted into and through a channel of the endoscope or other medical device and the endoscope or other device may abut the distal end 20 of the inner handle member 16.

The stylet 32 and the inner needle 30 may be made of, for example, stainless steel or any other material that is suitable for introduction into a patient. Preferably, the stylet 32 and/or the inner needle 30 are made of conductive material.

The inner needle 30 may be slidably extended through the outer needle lumen 34 of the outer needle 28. The outer needle 28 may be made of any material suitable for introduction into a patient, such as, for example, stainless steel. Preferably, the outer needle 28 is made of thermally and/or electrically conductive material. The outer needle 28 may be slidably extended through the sheath lumen 26 of the sheath 24. The sheath 24 may be made of material that is suitable for introduction into a patient and is preferably made of poly ether ether ketone, or a metal coated with Teflon® or the like. Preferably, the sheath 24 is made of an thermally and/or electrically insulative material.

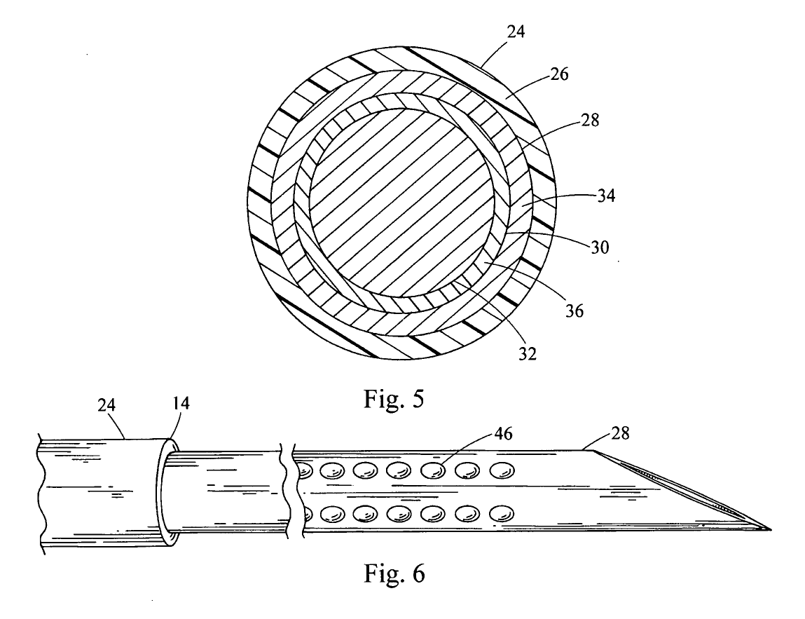

Referring briefly to FIG. 5, the generally coaxial relationship of the sheath 24, outer needle 28, inner needle 30 and stylet 32 is shown taken along line 5-5 of FIG. 1. Also shown in FIG. 5 are lumens 26, 34 and 36. It will be understood from FIG. 5 that, while a coaxial relationship is shown, the relationship among the various components of the needle system 10 need not be coaxial and may be offset relative to each other. It is also important to note that the space between, for example, the inner needle 30 and the outer needle 28 is sufficient enough such that the inner needle 30 may be slidably extendable through the outer needle 28. Likewise, the space between the outer needle 28 and the sheath 24 is sufficient enough such that the outer needle 28 is slidably extendable through the sheath 24. Moreover, the space between the stylet 32 and the inner needle 30 is enough such that the stylet 32 is slidably extendable through the inner needle 30, but sufficiently small enough to not allow material into the inner needle lumen 36 unless the stylet 32 is retracted within the lumen 36.

Referring back to FIGS. 1-3, the needle system 10 may also include a pin 44 disposed on the needle system near the distal end 20 of the inner handle member 16. The pin 44 may extend through the inner handle member 16 and make contact with the outer needle 28. Conductive heat may be sent through the pin 44 to the outer needle 28 by connecting a heat source to the pin 44, thereby heating the outer needle 28 throughout its entire length. The inner needle 30 as well as the stylet 32, by principles of conduction, will be heated as a result of electrical current being applied to the pin 44.

FIG. 6 is an embodiment illustrating the distal end 14 of the needle system 10 in the closed configuration that may include a plurality of indentations 46 disposed on the outer needle 28. The indentations 46 may also, or alternatively, be disposed on the inner needle 30. The indentations 46 may serve to produce an image in response to a sonic beam from imaging equipment. The image produced by the indentations may assist the operator in guiding the needle system 10 through a passageway of a patient. This methodology includes directing a sonic beam toward the passageway of the patient with the needle system 10 inserted therein and receiving an image from the indentations 46 of the outer needle 28. The material of the outer needle 28 for this embodiment of the invention, or the inner needle 30, if it contains indentations 46, is selected to have acoustic impedance different from that of the surrounding medium, e.g., body fluid from a patient or air which generally has an acoustic impedance of approximately 428 MKS rayls. A more detailed description of one example of a type of sonic imaging system is disclosed in U.S. Pat. No. 5,081,997, the contents of which are incorporated herein.

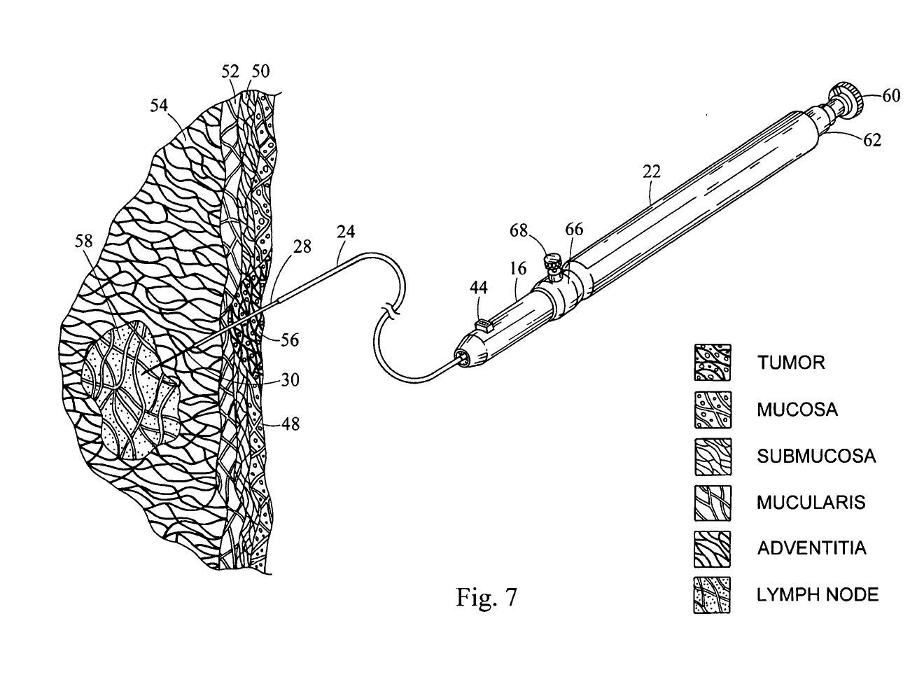

FIG. 7 illustrates a method of implementing a biopsy needle system 10 during, for example, fine needle aspiration. As an illustration, FIG. 7 represents the layers of the esophageal wall, where the present method may be implemented. It should be understood, however, that the applications of this method are not limited to the esophagus and can be applied anywhere in a patient where a tumor or other lesion obstructs a lymph node or access to a lymph node or a tumor or other lesion that obstructs a mass behind a patient's soft tissue or obstructs access to a mass behind a patient's soft tissue.

The layers of the esophageal wall are shown, namely, mucosa 48, submucosa 50, mucularis 52 and adventitia 54. Also shown are a tumor 56 and a lymph node 58. For illustration purposes, the lymph node 58 is obstructed or partially obstructed by the tumor 56 which may be identified by a physician or other medically trained persons using known means such as, for example, endoscopy, radiography and/or CT scans. In such a situation, one preferable way to biopsy the lymph node 58 would be to enter through the tumor 56 and into the lymph node 58 to retrieve a sample. By incorporating the advantages of the present system into the FNA procedure, the instances of tumor seeding are substantially prevented.

The inner needle 30 initially is retracted within the lumen 34 of the outer needle 28. Moreover, the outer needle 28 initially is retracted within the lumen 26 of the sheath 24. In other words, initially, the needle system is in the open configuration. The operator locates the area of the patient where the biopsy procedure is to occur and then applies electrical current to the pin 44, which then transfers heat to the outer needle 28 which may still be retracted within the lumen 26 of the sheath 24.

Through conduction, the inner needle 30 also is heated through application of the electrical current to the pin 44. The operator may then extend the outer needle 28, inner needle 30 and stylet 32 axially beyond the distal end 38 of the sheath 24 to expose a portion of the outer needle 28 prior to contacting the tumor 56. As described above, a user of the needle system 10 can advance the outer needle 28, inner needle 30 and the stylet 32 to a desired position relative to the sheath 24. At this point, although the inner needle 30 and stylet 32 axially extend beyond the distal end 38 of the sheath 24, the inner needle 30 is still retracted within the outer needle lumen 34.

The operator then may advance the heated outer needle 28 through a layer of mucosa 48 into the tumor 56. The outer needle 28 acts to cauterize a path through the tumor 56 such that the tumor cells will be substantially prevented from seeding to other areas of the patient. Another advantage of cauterization would be to minimize bleeding during the procedure. Once the outer needle 28 has passed through the tumor 56 and a cauterized path has been made, the electrical current is no longer applied to the pin 44. This allows the outer needle 28, the inner needle 30 and the stylet 32 to cool down prior to further advancement through the tissues of a patient.

After being allowed to cool down, the needle system 10 is then further advanced through the remaining esophageal layers to approach the lymph node 58. The operator then advances the needle system 10 such that the inner needle 30 pierces the lymph node 58. As described above, the inner needle 30 and the stylet 32 may extend axially beyond the distal end of the outer needle 28. Because the inner needle 30, until this point, has been retracted within the outer needle 28, the inner needle 30 did not come directly in contact with the tumor 56. Once the inner needle 30 is advanced within the lymph node 58, the stylet 32 may be retracted and the inner needle 30 may be further advanced such that a sample may be gathered from the lymph node 58. It should also be understood that the inner needle 30 and the stylet 32 may be further retracted in the outer needle 28 and the outer needle 28 may be advanced to collect a sample.

Once the sample is obtained, the operator may retract the inner needle 30 back within the lumen 34 of the outer needle 28. Likewise, the outer needle 28 may be retracted back within the lumen 26 of the sheath 24. The sheath 24 then may be advanced out of the patient and the sample preserved for storage and/or analysis.

It should be understood that insertion points and layers of the esophagus are provided by way of example only and the description of the method should not be limited to the examples herein described. The advantages of the present system may be realized at any location in a patient where a tumor cell is obstructing access to a lymph node or other target tissue for biopsy. It should also be understood that the invention is not limited to tumor cells that obstruct a lymph node or other target tissue. Rather, the present system may be applied to any obstructing mass that has the potential to seed cells, the prevention of which would be advantageous to the patient.

The foregoing detailed description provides exemplary embodiments of the invention and includes the best mode for practicing the invention. These embodiments are intended only to serve as examples of the invention, and not to limit the scope of the invention in any manner.

1. A method for performing fine needle aspiration of a target tissue, the method comprising the sequential steps of:
providing a needle system;
applying heat to the needle system prior to passing through a tumor or other lesion;
advancing the heated needle system through the tissue layers of a patient and through the tumor or other lesion;
cooling the needle system;
collecting a sample from a lymph node or other target tissue by advancing the needle system into the lymph node or the other target tissue; and
withdrawing the sample and the needle system from the patient.

2. The method of claim 1 wherein the needle system comprises a stylet movably disposed within an inner needle, and the step of advancing the heated needle system through the tissue layers of a patient and through the tumor or other lesion is performed while the stylet is retracted within the inner needle.

3. A biopsy needle system, comprising:
an inner handle member having proximal and distal ends;
an outer handle member slidably disposed on the inner handle member;
an elongate sheath attached to the inner handle member and axially extending beyond the distal end thereof, the sheath defining a sheath lumen;
an outer needle attached to the outer handle member and extending through the sheath, the outer needle being slidably disposed within the sheath;
an inner needle attached to the inner handle member and slidably disposed within the outer needle;
a stylet slidably disposed within the inner needle; and
means for heating the needle system.

4. The biopsy needle system of claim 3 wherein the needle system further comprises a connector, configured to accept instruments that aid in the gathering of the sample, and a stylet cap fixably attached to the stylet and disposed on the connector.

5. The biopsy needle system of claim 3 wherein there is slidable movement of the outer handle member relative to the inner handle member which controls the relative positioning of the outer needle, inner needle and stylet.

6. The biopsy needle system of claim 3 further comprising a series of gradations disposed on the inner handle member which correspond to a predetermined length by which the outer needle, the inner needle and the stylet extend axially beyond the distal end of the sheath.

7. The biopsy needle system of claim 3 further comprising a plurality of indentations disposed on the outer needle.

8. The biopsy needle system of claim 3 further comprising a plurality of indentations disposed on the inner needle.

9. The biopsy needle system of claim 3 further comprising an intermediate handle member.

10. The biopsy needle system of claim 3 wherein the stylet is made of stainless steel.

11. The biopsy needle system of claim 3 wherein the distal end of the inner handle member is fixably attached to a medical device.

12. The biopsy needle system of claim 3 where the distal end of the inner handle member is fixably attached to an endoscope having at least one channel and where the elongate sheath is slidably disposed in the channel of the endoscope.

13. A method for performing fine needle aspiration of a target tissue, the method comprising the sequential steps of:
providing a needle system, the needle system comprising an outer needle, an inner needle, and a stylet;
applying heat to the needle system through a pin in contact with the outer needle;
advancing the heated needle system through the tissue layers of a patient and through a tumor or other lesion that is obstructing or partially obstructing a target tissue;
cooling the needle system;
collecting a sample from the target tissue by advancing the needle system into the target tissue; and
withdrawing the sample and the needle system from the patient.

14. The method of claim 13 where the needle system further comprises an outer handle fixably attached to the outer needle; an inner handle fixably attached to the inner needle and slidably disposed within the outer handle.

15. The method of claim 13 where the pin is connected to an external source for delivering heat to the pin.
